(19) United States
(12) Patent Application Publication
(10) Pub. No.: US 2005/0251073 A1
(43) Pub. Date: Nov. 10, 2005

(54) Device for foot stabilization

(75) Inventor: Ivar E. Roth, Newport Beach, CA (US)

Correspondence Address: DR. ROTH'S FOOTCARE PRODUCTS LLC, 1012 BRIOSO DRIVE SUITE 105, COSTA MESA, CA, 92627, US

(73) Assignee: Dr. Roth's Footcare Products LLC, Newport Beach, CA (US)

(21) Appl. No.: 10/841,928

(22) Filed: May 10, 2004

(51) Int. Cl. A61F 5/00; A43B 13/38

(52) U.S. Cl. 602/8

(57) ABSTRACT

A supportive device for the transverse metatarsal arch of the foot is comprised of a fabric-coated elasticized strap enclosing a specially designed pad of rubberized material. This strap is wrapped snugly around the transverse metatarsal arch with the enclosed pad strategically placed to provide optimum arch support, metatarsal relief and secured by a hook and loop type fastener strip.

### BACKGROUND OF THE INVENTION

1. Field of the Invention

The invention relates generally to orthopedic devices for the foot and more specifically to supporting the transverse metatarsal arch of the foot in a simple, unobtrusive manner.

2. Description of the Prior Art

There are two anatomical arches of the foot, the transverse (metatarsal) arch and the longitudinal arch. The invention is concerned only with the transverse metatarsal arch. Supporting this arch by mechanical means is an indicated and accepted modality for several conditions of the foot.

Metatarsaligia and Capsulitis are conditions which involves pain of the metatarsal heads. This may occur by a dropping down of the actual metatarsal head(s) or an elongated metatarsal, injury or prominent metatarsal head because of dorsal foot contractions. This invention is a pressure relief device which aids in decreasing the pressure on the metatarsal heads.

An additional, yet significant cause is atrophy of the fat underlying the metatarsal heads. As one ages the fat pad atrophies thus losing its protective component thus exposing the underlying metatarsal heads to injury this invention is a pressure relief device which aids in decreasing the pressure on the metatarsal heads.

Splaying of the transverse metatarsal arch results in undesired increased motion and instability of the transverse metatarsal arch. This inventions helps support and maintain the transverse metatarsal arch by use of the supportive adjustable band and the metatarsal pad which helps to maintain the transverse metatarsal arch.

Any device that can stabilize and support the transverse metatarsal arch will allow its wearer to stand or walk in greater comfort and reduce the chances of further pathology. At present, the most widely accepted effective and long term method of providing this support is by means of molded arch supports. Commonly called, arch supports or orthodic devices. These devices are made from a wide range of materials and may be customized by a physician at the high monetary end of the spectrum or purchased as an over the counter commercial item from many outlets at the low end of the spectrum. They are durable and provide consistent results over time.

There are major disadvantages besides cost: 1) the space they take in shoes, 2) not all types of shoes accommodate them (open types do not): 3) their impractibility for certain athletes that require little or no foot wear (such as dancers or gymnasts.) 4) generally over the counter orthodics do not specifically address the transverse metatarsal arch.

Strapping and padding the foot with various types of adhesive tape to maintain optimal function has also been a mainstay method of addressing the problem of metatarasaligia et al . . . While this method is quite practical for short term use by physicians and professional trainers, it has numerous draw backs. For example: 1) for optimal results it should be applied by an professional, which rules out self-application, 2) it stretches and loses its effectiveness quickly (two or three days is usually the maximum limit for wear.) 3) it can be used only once, 4) no adjustment is possible after the initial application. Finally, 5) adhesive material may be irritating to the skin, can facilitate fungal infections and has the potential for allergic reactions.

Other non-orthodic devices which support the transverse metatarsal arch in varying ways are:

The Metatarsal Arch Cushion Support (U.S. Pat. No. 3,086,520) incorporates a pad with an encircling elasticized band, however there are two major differences in concept between this device and the present device 1) “the pad is to be positioned beneath the metatarsal arch with the high part of the body portion under the most aggravated part of the arch.” In comparison the present device utilizes the pad whereby the metatarsal shaft and neck lie on the high part of the pad therefore suspending the metatarsal heads past the device: thus acting as a pressure relieving device for the metatarsal heads. 2) its encircling band is closed and non-adjustable yet is described as one size fits all; this translates to minimal support due to its inability to specifically adjust to the varying foot sizes, were as described below the strap is open and adjustable to ensure proper fit, which acts as a compressive support to the transverse arch and consequently provides comfort. 3) Its bulk could make wearing in most shoe either difficult or impossible. Whereas the present device is minimized to its essential anatomical necessity thus reducing bulk. 4) The device has stitched seems which may become a problem for potential skin irritation. Whereas the present device has no stitched seam therefore eliminating this concern.

Arch Support (U.S. Pat. No. 1,492,514), has incorporated with it a wide encircling band and removable and adjustable arch pads. The removable pads have the potential to shift position thereby becoming less effective. Determining which pad thickness is most appropriate may require professional advice. Whereas the present device has permanently affixed pre-positioned pads and a thinner dorsal band which is less obtrusive and allows for easier shoe fitting.

What is needed is a device which is more effective, simpler and easy to apply. The present invention satisfies the need for further improvements in this area.

### BRIEF SUMMARY OF THE INVENTION

The present invention provides a new and simplified apparatus for supporting the transverse metatarsal arch and elevating the metatarsal heads. The key element in this invention is the specially designed strategically placed, rubberized, resilient pad. The convex side of this pad presses up against the transverse metatarsal arch, opposing its collapse, by supporting the metatarsal shaft and neck and elevating the metatarsal head. The pad contained within the strap, is further aided in stabilizing the transverse metatarsal arch by the pressure of the circumferential wrapping around the arch by the strap itself.

Supporting and elevating the metatarsal shaft and neck, and elevating the metatarsal head will accomplish two main goals: 1) in feet with contractures there will be lessened contractures due to the windless effect (that is, by pushing up on the dropped bones, there will be a reciprocal downward force movement on the contracted dorsal foot elements.) 2) Metatarsal head pain will be alleviated due to the repositioning afforded by the pad support. In comparison, to the other apparatuses/devices previously described, this invention affords the following advantages: 1) Increased comfort to the wearer and better shoe fit due to the slim nature of the device. 2) Simpler self-application because of the design concept. 3) Can be used by physicians as a temporary device in place of taping and strapping. 4) over the counter availability will enhance early self treatment which may avoid costly medical treatment; has the added benefit of use in barefoot activities further enhancing its beneficial effects.

In particular the invention is a device for supporting the transverse metatarsal arch and reducing the pressure to the metatarsal heads. The device comprises substantially non-extensible, elongated, foam-covered laminate for encircling the transverse metatarsal arch and to, in part stabilize it. The laminate tapers away from that portion fixed to the pad so that when it is applied to the foot the tapered portion extends across the top of the foot opposite the arch and is less obtrusive.

A substantially elastic elastomeric pad, which regains substantially all of its original thickness after repeated compressions, is fixed to the laminate for absorbing energy and defending against shock. The laminate and pad when applied to the foot disposes the pad at a predetermined position under the transverse metatarsal arch to decrease the amount of pressure on the metatarsal heads. The pad has a convex surface. The convex surface extends outwardly from the laminate so that the convex surface extends into the arch at the predetermined position.

A hook-type fastener, having a hook portion fixed to one end of the laminate with the an eye portion being the exterior surface of the laminate, secures the laminate and pad to the foot at the predetermined position, yet allows for variations in size.

The fastener and pad are fixed to the laminate without stitching so that comfort and thinness is enhanced.

The laminate and the fastener each have a predetermined length to allow a range of fastening positions of ends of the laminate to each other. The length of the laminate and the fastener being chosen to accommodate all possible sizes of the foot.

The laminate, pad and fastener have a latitudinal axis and wherein said strap and said fastener are symmetric about said latitudinal axis of said strap fastener, and, when the device is worn said pad is symmetrical about said longitudinal axis of said foot so that said device is equally applicable to a left and right foot.

These advantages and other aspects of this invention will be apparent from the detailed description and drawings which follow wherein like elements are referenced by like numerals.

### BRIEF DESCRIPTION OF THE DRAWINGS

FIG. 1 is a perspective view of the device in an open and slightly curved configuration to show its general dimensions. The shaded portion of the Velcro-type fastener (12) indicates that this portion contains the hooks which fasten to the exterior surface of the strap when applied. The outline (18) indicates the pad placement. Throughout the specification, “exterior” or “outside” is meant to refer to that portion of the device which is exposed or on the outside of or not touching the foot after the device is applied to the foot as intended. “Interior” or “inside” is meant to refer to that portion of the device which is not exposed or on the inside of or touching the foot after the device is applied to the foot as intended.

FIG. 2 is an outside plan view of the device showing additional general dimensions. The top portion (14) of the Velcro-type fastener above the dotted line indicates that portion which is adhesively or otherwise bonded to the exterior surface of the device in a non-stitched fashion. The dotted line (13) indicates the underlying narrow end of the device itself.

FIG. 3 is a cross-sectional view of the device showing the various layers which make up the device (not necessarily to scale).

FIG. 4 is an inside plan view of the device indicating its general shape and proportions, with the shaded portion of the VELCRO fastener (12) indicating the hook portion and the dotted outline indicating the pad placement.

FIG. 5a is top plan view of just the rubberized pad in the device.

FIG. 5b is cross-sectional view of just the rubberized pad in the device.

FIG. 6 is a phantom view of the foot showing the placement of the device and the orientation of the pad proximal to the metatarsal head region.

FIG. 7 is a parallel view of the foot with the device in its final, secured position.

The invention and its various embodiments may now be understood by turning to the following detailed description.

### DETAILED DESCRIPTION OF THE PREFERRED EMBODIMENTS

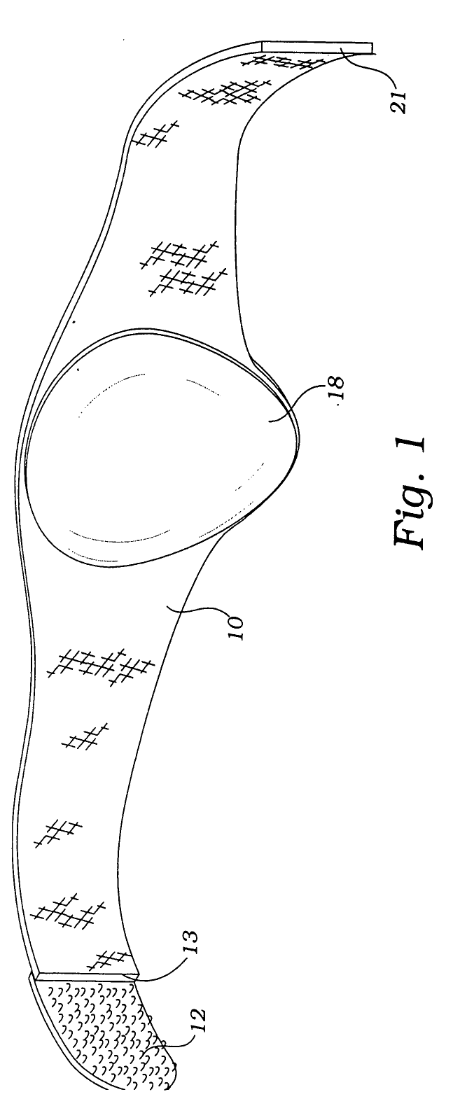

A supportive device for the transverse metatarsal arch of the foot is comprised of a fabric-coated elasticized strap enclosing a specially designed pad of rubberized material. This strap is wrapped snugly around the transverse metatarsal arch with the enclosed pad strategically placed to provide optimum arch support and secured by a VELCRO fastener strip. A strap of fabric coated foam material (general dimensions 9″ by 3″), is provided with a specially designed, triangular-shaped, rubberized pad (general dimensions 3″ by 2.5″ and 3/16″ thickness). The narrow end of the pad points perpendicular to the axis of the strap. The flat side of the pad is bonded to the inside of the device at center of the strap. The convex side of the pad is in contact with the foot when the device is applied. A VELCRO fastener (general dimensions 1.5″ by 1″) secures the device to the foot. FIG. 1 shows only one half of the Velcro-type fastener (shaded area) 12, which is the portion which when pressed into the exterior surface of laminate body 10, fastens the device to the foot, which exterior surface acts as the eye portion of the fastener.

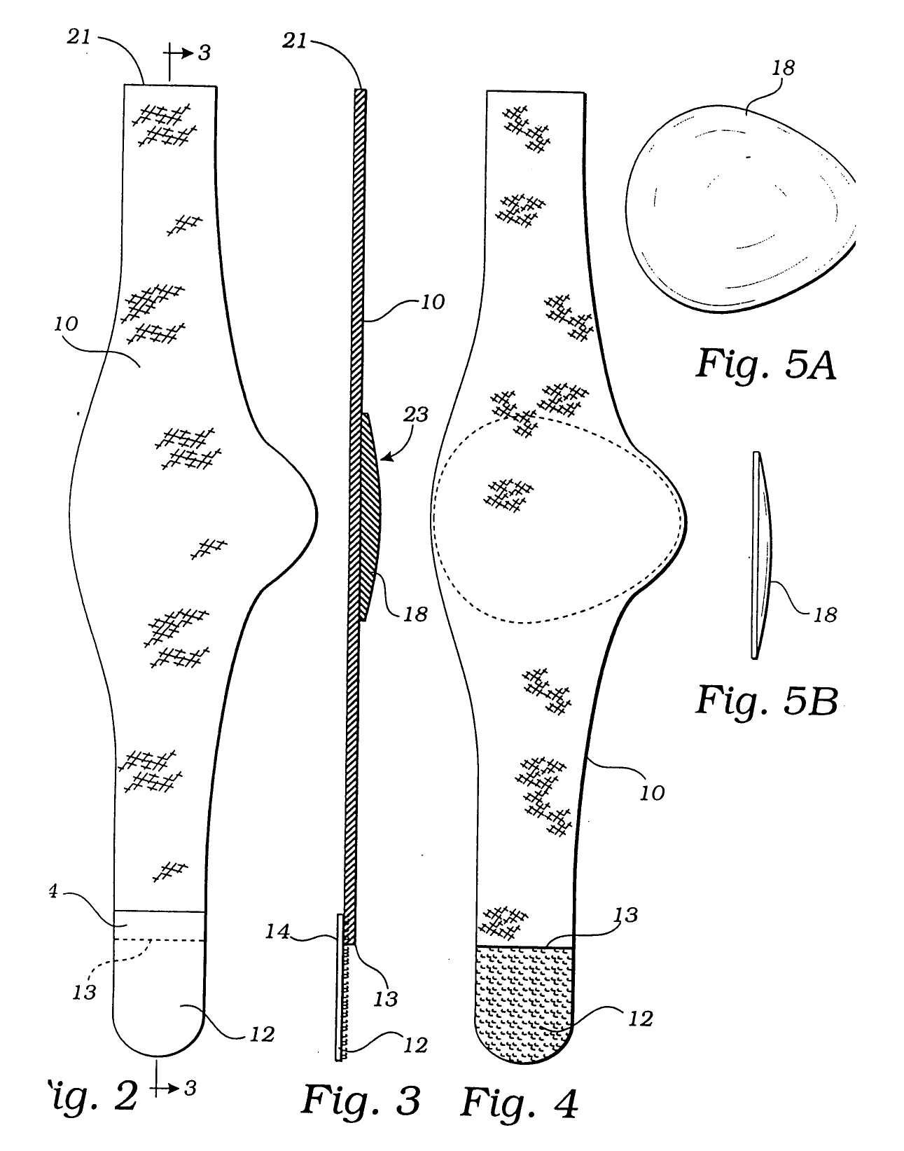

FIG. 1 shows in perspective view a flexible strip of fabric-coated foam material body 10 having in the illustrated embodiment general dimensions 9″ by 3″, with a specially designed, triangular-shaped, rubberized resilient pad 18, which again in the illustrated embodiment measures about 3″ in the latitudinal direction of device by 2.5″ in an orthogonal direction and 3/16″ in thickness. Pad 18's outer outline is curvilinear and is chosen to comfortably conform to the cavity of the arch. The narrow end of the pad 19 points outward from the center of said device. The flat side of pad 18 is bonded to the inside of the device at the center portion of device. The convex side 23 of the pad 18 as shown in FIG. 3 is in contact with the foot when the device is applied as intended. A plastic VELCRO fastener 12, which in the illustrated embodiment has dimensions 1.5″ by 1″, is adhesively fixed, stitched, heat bonded, or imbedded in the narrow end 13 of the strip to secure it. Fastener 12 may by fixed to the exterior, or interior surface of body 10, or have a thin split attaching portion which sandwiches body 10 and is fixed to both its interior and exterior surfaces. Exterior attachment is shown in FIG. 3 only as an illustration.

FIG. 2 shows a outside plan view of the device wherein surface of body 10 has a nappy or felt-like surface texture to function as the eye portion of the Velcro fastener and is provided near narrow end 13 with a VELCRO fastener.

FIG. 3 is a cross-sectional view taken through lines 3-3 of FIG. 2 and illustrates that the VELCRO fastener is adhesively fixed to the device at or near the opposite end 13 with a portion of the VELCRO fastener 12 overlapping a portion of the adjacent outside surface of body 10. Pad 18 is adhesively secured to the approximate center of the interior surface of body 10.

Pad 18 is shown in isolation of the other elements of the device in FIG. 5a in plan view and in side elevational view in FIG. 5b. Other shapes and proportions may be utilized other than those suggested by the illustrations without departing from the scope of the invention.

There are two embodiments, the Dress/Regular model and the Sports/Heavy Duty model, but one size fits all. The only difference between these embodiments is the thickness of the strap material. The Dress/Regular model uses a thinner material and the Sports/Heavy Duty model uses a heavier material.

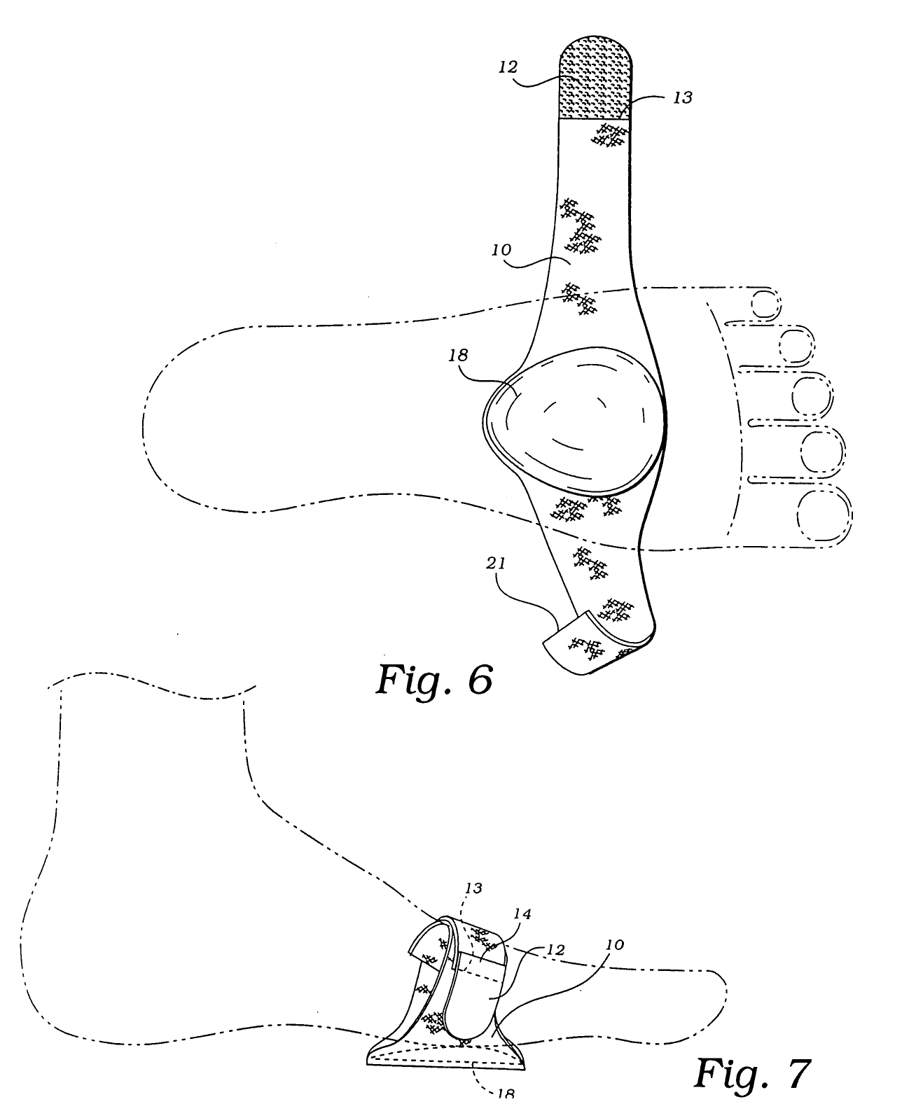

The structure of the device now having been described, consider how it is applied to or wrapped around the foot. While aligning and placing pad 18 just proximal to the metatarsal heads and holding end 21 of the device in one hand, end 13 is brought over the top of the foot with the other hand as shown in FIG. 6. After sufficient pressure is applied to ensure a snug fit with the arch, the hook side of the VELCRO fastener 12 on the strap end 13 is pressed into the exterior surface of body 10 of the device to secure it to the foot as shown in FIG. 7. Thus the only material in contact with the skin is the smooth interior surface of the device.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following claims.

The words used in this specification to describe the invention and its various embodiments are to be understood not only in the sense of their commonly defined meanings, but to include by special definition structure, material or acts beyond the scope of the commonly defined meanings. The definitions of the words or elements of the following claims are, therefore, defined in this specification to include not only the combination of elements which are literally set forth, but all equivalent structure, material or acts for performing substantially the same function in substantially the same way to obtain substantially the same result.

In addition to the equivalents of the claimed elements, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. A device for stabilizing either a left or right foot by simultaneously supporting the transverse metatarsal arch and off-loading the metatarsal heads comprising of: a flexible strap of an elastic material having first and second ends for wrapping around the foot; a fastener for temporarily securing ends of said strap together on the foot; and an elastomeric pad formed of a material that substantially regains its original thickness after compression and has an edge surface and inwardly tapered sides extending from said edge surface generally in the direction of a longitudinal axis of the pad toward a narrow end of the pad, said pad being fixed to an inner surface of said strap for absorbing energy and defending against shock, at a position on said strap in the center of said strap and oriented with the narrow end of said pad facing perpendicular to either end of the strap such that when said strap and pad are applied to the foot, and said pad is disposed under said transverse metatarsal arch proximal to the metatarsal heads the longitudinal axis of said pad is approximately parallel and centered to the longitudinal axis of the transverse metatarsal arch of the foot, and said tapered portion of said pad is disposed from the longitudinal axis of the foot toward the heel.

2. The device of claim 1 wherein said strap is a foam covered laminate thus having a nonslip surface.

3. The device of claim 1 wherein said strap and said fastener have a latitudinal axis and wherein said strap and said fastener are symmetric about said latitudinal axis of said strap and fastener, and, when the device is worn, said pad is symmetrical about said longitudinal axis of said foot so that said device is equally applicable to a left and right foot.

4. The device as in claim 1, wherein the entirety of said strap is made of a flexible material.

5. The device as in claim 4, wherein said strap is formed of a coated foam material.

6. The device as in claim 1, wherein said strap is tapered towards its ends.

7. The device as in claim 1, wherein said strap is of maximum width at its center and tapers toward said first and second ends, and said pad is located on said strap at a region of said maximum width.

8. A device as in claim 1, wherein said strap and pad have lateral symmetry about the latitudinal axis of the strap.

9. A method of treating a foot for metatarsalgia, capsulitis by simultaneously supporting the transverse metatarsal arch and off-loading the metatarsal heads, comprising the steps of wrapping the foot with an elastic strap, the strap bearing an elastomeric pad formed of a material that substantially regains its original thickness after compression and has an edge surface and inwardly tapered sides extending from said edge surface generally in the direction of a longitudinal axis of the pad toward a narrow end of the pad, said pad being fixed to an inner surface of said strap for absorbing energy and defending against shock; and positioning on said strap on the foot such that said pad is disposed under the transverse metatarsal arch proximal to the metatarsal heads said pad is approximately parallel and centered to the longitudinal axis of the transverse metatarsal arch of the foot, and said tapered portion of said pad is disposed from the longitudinal axis of the foot toward the heel to support the transverse metatarsal arch and off-load the metatarsal heads.
